(19) United States
(12) Patent Application Publication
(10) Pub. No.: US 2007/0073304 A1
(43) Pub. Date: Mar. 29, 2007

(54) Femur alignment guide device

(76) Inventors: Jai-Gon Seo, Seoul (KR); Eun-Suk Seo, Seoul (KR); Chong-Bum Kim, Seoul (KR)

Correspondence Address: PARK LAW FIRM, 3255 WILSHIRE BLVD, SUITE 1110, LOS ANGELES, CA, 90010, US

(21) Appl. No.: 11/517,650

(22) Filed: Sep. 8, 2006

(30) Foreign Application Priority Data
Sep. 9, 2005 (KR) 10-2005-84056

(51) Int. Cl. A61F 5/00

(52) U.S. Cl. 606/86

(57) ABSTRACT

A femur alignment guide device, which can be mounted on the femur vertically to the mechanical axis based only on the mechanical axis without measuring the angles between the mechanical and anatomical axes which are different according to the photographing angle, is disclosed. The femur alignment guide device comprises a body having a pair of mounting holes formed transversely for mounting a femur front bone cutter and a femur bottom end cutter, and a pair of pinholes formed vertically for inserting the pin driven into the femur bottom end; and a pair of top surface adjusting means mounted on both sides of the body so as to make ascent and descent possible.

### CLAIMING FOREIGN PRIORITY

The applicant claims and requests a foreign priority, through the Paris Convention for the Protection of Industrial Property, based on patent applications filed in the Republic of Korea (South Korea) with the filing date of Sep. 9, 2005 with the patent application number 10-2005-84056 by the applicant, the contents of which are incorporated by reference into this disclosure as if fully set forth herein.

### BACKGROUND OF THE INVENTION

1. Field of the Invention

The present invention relates to a femur alignment guide device.

2. Description of the Related Art

Knee arthroplasty has become one of the most commonly performed surgeries, in which a damaged or deformed knee joint due to congenital deformation, traumatic injuries, diseases, degenerative arthritis, etc. is removed and replaced with an artificial joint. In such a knee arthroplasty, accurate cutting of the damaged knee joint is very important to prevent side effects after surgery and prolong the life of the replaced artificial knee joint.

Here, in order to cut the knee joint of the patient and perform a knee arthroplasty without side effects, the femur and tibia should be cut vertically with respect to a mechanical axis so that the bottom end cut surface of the femur and the top end cut surface of the tibia are maintained in parallel each other, as shown in FIG. 1a. Bellow, the present detailed description will be made centering on a cut of the bottom end of femur related to the present invention.

The mechanical axis of femur refers to an axis connecting the center of the femur head and the center of the femur bottom end each other as shown in FIG. 1b. As it cannot be seen from outside, image photographing is done with X-ray to determine it. And an anatomic axis is an axis of a bony canal for inserting a bone cavity bar.

Meanwhile, in order for a femur to be cut vertically to the mechanical axis, a femur bottom end cutter should be mounted on the femur vertically to the mechanical axis, and a femur alignment guide device is used for this.

As shown in FIG. 2a, a conventional guide device includes a body 10 having a top surface 11 as a support surface for supporting the bottom end of femur, a through hole 12 formed in a vertical direction near the center of the top surface 11, and a predetermined angle graduation portion 13 formed at the lower part of the body 10; and a hollow 22 insert 20 which is assembled to the angle graduation portion 13 of the body 10 and having an indicator 21 which is pivotly inserted and assembled into the through hole 12 of the body 10. Reference numeral 14 is a hole for fitting a femur front bone cutter and femur bottom end cutter, and reference numeral 15 is a pinhole for inserting the pin driven into the bottom end of the femur.

In use, as shown in FIG. 2b, first obtain the angle θ between the mechanical and anatomical axes by image photograph taken with X-ray, and turn and fix the insert 20 inserted in the body corresponding to this angle. Next, insert bone cavity bar B into the insert 20 to advance it into the bone cavity so as to pass the pin (P-1, see FIG. 2c) through the pinhole 15, while the bottom end of the femur is supported by the top surface 11 of the body 10. After that, drive the pin P-1 into the bottom end of femur to mount the femur alignment guide device on the bottom end of femur.

Subsequently, as shown in FIG. 2c, insert the femur front bone cutter 30 in the mounting hole 14 of the femur alignment guide device to cut the femur front bone, and then, as shown in FIG. 2d, insert the femur bottom end cutter 40 in the mounting holes 14 of the femur alignment guide device. After that, fix the femur bottom end cutter 40 to the femur with a pin P-2, and finally remove the femur alignment guide device. Subsequently, as shown in FIG. 2e, insert a cutter C into the bone-cutting gap of the femur bottom end cutter 40 to cut the bottom end of the femur.

However, the conventional femur alignment guide device brings about the following problems.

First, in the conventional art, the angle θ between the mechanical and anatomical axes is obtained by X-ray taken image photograph before mount the femur alignment guide device on the femur based on the angle θ. But, as shown in FIG. 3a, the angles at this time can be measured in different angles θ, θ′ according to the angle of photographing the femur. Therefore, if the guide device is mounted on the femur by adjusting the angle of the insert according to θ′, which is an angle different from θ, the normal photographing angle, the guide device cannot be mounted vertically to the mechanical axis as much as the error angle θ-θ′, so eventually there is a problem of the femur bottom end not being cut vertically along the mechanical axis.

Second, in the conventional case, regardless whether the guide device is mounted according to the normal photographing angle θ or not, if the protruding portion of one side of the femur bottom is damaged more than the protruding portion of the other side, as shown in FIG. 3b, the top surface of the body cannot fully support the protruding portions of both sides, supporting only the protruding portion of one side. Especially, in the process of driving the pin, the guide device is mounted in deviation from the mechanical axis, so the bottom end of femur is not cut vertically along the mechanical axis, causing side effects to knee arthroplasty.

Third, in order to use the conventional guide device, after inserting the insert, the bone cavity bar had to be put into the bone cavity through an input hole punched at the bottom end of the femur so as to be connected to the bone cavity. But if the input hole is not punched in the correct location at the bottom end of the femur but punched in deviation, the bone cavity bar is inserted in misalignment with the anatomic axis, so eventually there is a problem that the guide device cannot be mounted vertically along the mechanical axis. Another problem is that due to input of bone cavity bar the bone cavity is damaged or recovery time following replacement surgery is prolonged. A further problem is that if a hip joint replacement is inserted in the bone cavity, the very insertion of bone cavity bar is impossible due to occlusion of bone cavity.

### SUMMARY OF THE INVENTION

Therefore, it is an object to provide a femur alignment guide device which can be mounted on the femur vertically along the mechanical axis based only on the mechanical axis without measuring the angles between the mechanical and anatomical axes that are different according to photographing angles.

Another object of the present invention is to provide a femur alignment guide device which can support the protruding portions of both sides of the femur bottom end wholly in close contact.

Yet another object of the present invention is to provide a femur alignment guide device, which can be mounted on the femur vertically to the mechanical axis without putting the bone cavity bar into in the bone cavity, so problems due to forming an input hole at the bottom end of femur can be solved, and even if a hip joint replacement is inserted in the bone cavity, a bone cavity bar can be inserted into the bone cavity easily.

In accordance with the present invention, there is provided a femur alignment guide device comprising: a body having a pair of mounting holes formed transversely for mounting a femur front bone cutter and a femur bottom end cutter, and a pair of pinholes formed vertically for inserting the pin driven into the femur bottom end; and a pair of top surface adjusting means mounted on both sides of the body so as to make ascent and descent possible.

In accordance with an aspect of the present invention, the top surface adjusting means comprises a stepped portion formed on both sides of the body of the guide device and having a slide slot with a predetermined depth; a slider moving up and down by a leg inserted into the slide slot; a horizontal screw hole formed so as to be penetrated perpendicularly to the slide slot from the back of the body; and a fixing screw inserted into the horizontal screw hole to press the leg of the slider to fix the slider against the body.

In accordance with another aspect of the present invention, the top surface height adjusting means comprises a recess formed on both sides of the body of the guide device and having a vertical screw hole with a predetermined depth; and an adjusting knob screwed to a vertical screw hole of the recess to slide up and down.

### BRIEF DESCRIPTION OF THE DRAWINGS

Other objects and aspects of the present invention will become apparent from the following description of embodiments with reference to the accompanying drawings in which:

FIGS. 1a and 1b are drawings for describing the mechanical axis in conventional knee joint arthroplasty;

FIG. 2a is a perspective view showing a conventional femur alignment guide device;

FIGS. 2b through 2e are drawings for describing use state of the conventional femur alignment guide device;

FIGS. 3a and 3b are drawings for describing problems of the conventional femur alignment guide device;

FIG. 4 is a femur alignment guide device according to a preferred embodiment of the present invention;

FIG. 5 is a lateral sectional view of a top surface height adjusting means shown in FIG. 4;

FIG. 6 is a perspective view of a modified embodiment of the guide device shown in FIG. 4;

FIGS. 7a and 7b are drawings for showing how to use the guide device shown in FIG. 4;

FIG. 8 is a perspective view showing a guide device according to another embodiment of the present invention;

FIG. 9 is a sectional view of the top surface height adjusting means shown in FIG. 8;

FIG. 10 is a perspective view showing a modified embodiment of the guide device shown in FIG. 8;

FIG. 11 is a drawing for describing how to use the guide device shown in FIG. 8.

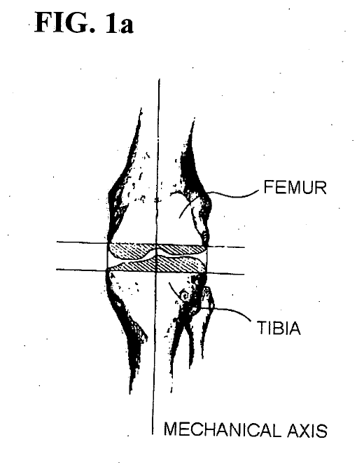

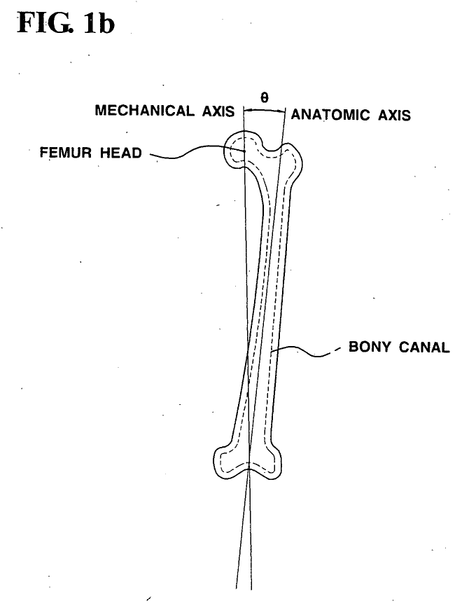

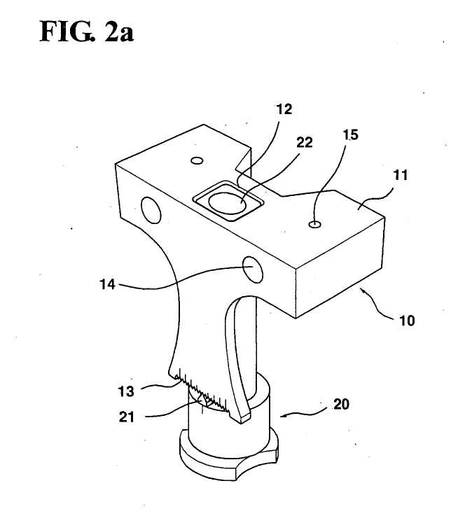

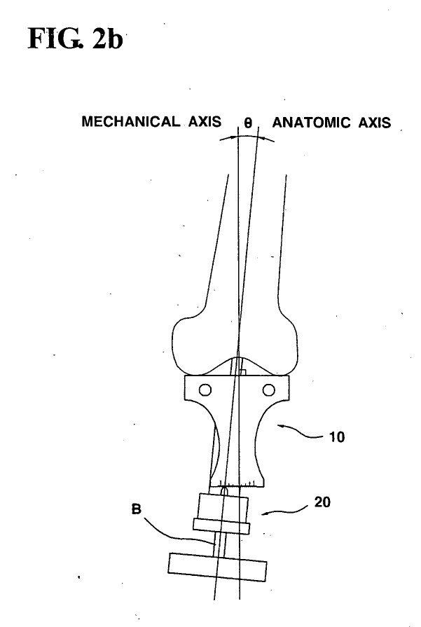

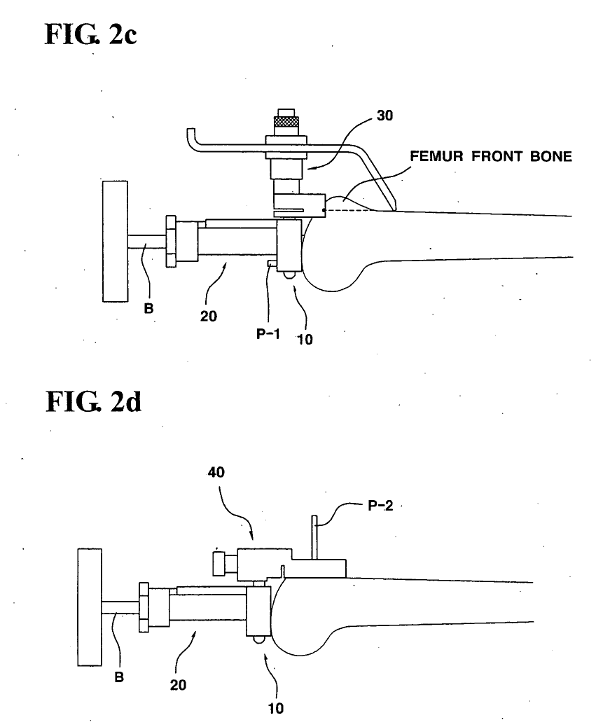

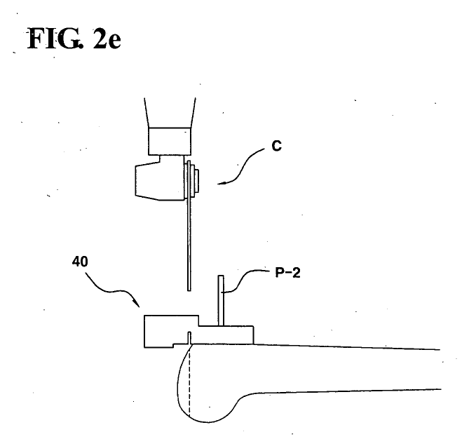

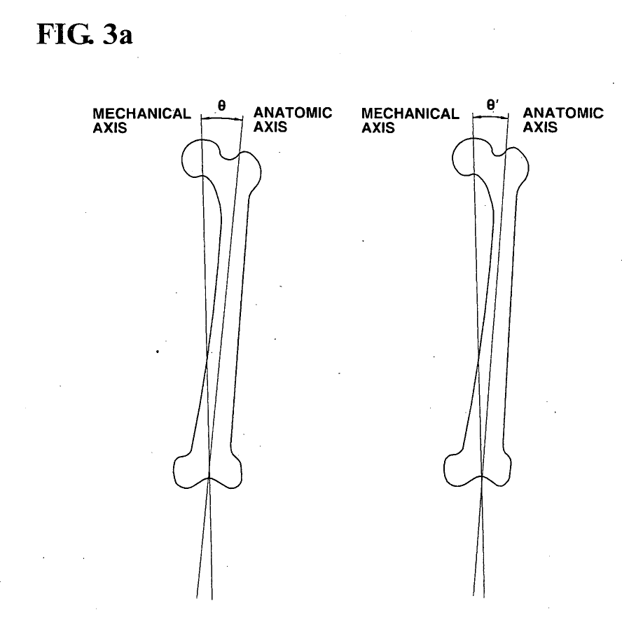

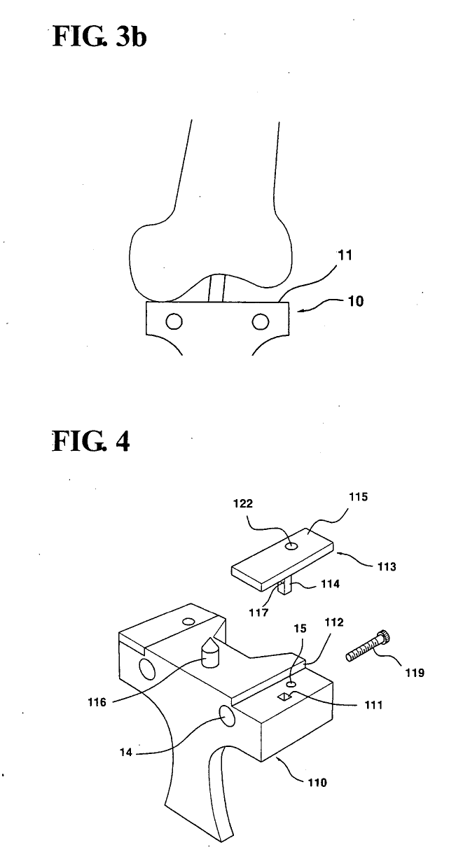

### DETAILED DESCRIPTION OF THE INVENTION

Below will be described in more detail a femur alignment guide device according to the present invention with reference to the accompanying drawings.

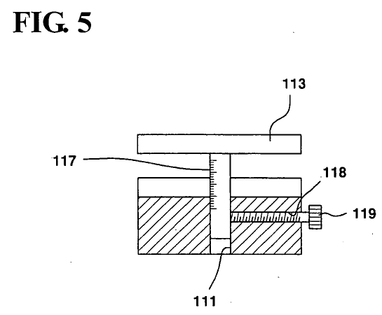

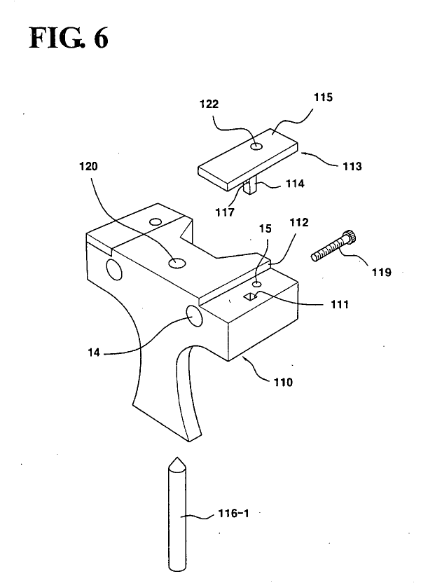

FIG. 4 is a perspective view showing a femur alignment guide device according to a preferred embodiment of the present invention, and FIG. 5 is a lateral sectional view of a top surface height adjusting means shown in FIG. 4, and FIG. 6 is a perspective view showing a modified embodiment of the guide device shown in FIG. 4.

As shown in these drawings, the guide device according to the present invention generally comprises a body 110 and a pair of top surface height adjusting means mounted on both sides of the body 110 such that they can ascend and descend.

The body 110 includes, as usual, a pair of mounting holes 14 formed transversely for mounting a femur front bone cutter 30 and a femur bottom end cutter 40, and a pair pinholes 15 formed vertically for inserting the pin P-1 driven into the bottom end of femur.

And, the top surface height adjusting means includes stepped portions 112 formed on both sides of the body 110 with a predetermined depth; a slider 113 sliding up and down by a leg 114 inserted into a slide slot 111; a horizontal screw hole 118 formed on the back of the body 110 so as to communicate perpendicularly to the slide slot 111; and a fixing screw 119 screwed to the horizontal screw hole 118 to fix the leg 114 of the slider 113 against the body 110.

The stepped portion 112 is formed by stepped machining the top surface of the body 110 at a predetermined depth, and the middle portion of the stepped portion 112 has the slide slot 111 formed for inserting the leg 114. Accordingly, the leg 114 of the slider 113 is inserted into the slide slot 111 and then the slider 113 is placed on the stepped portion 112. Here, the top surface 115 of the slider 113 becomes a support surface for supporting the bottom end of femur. And, the back of the body 110 is provided with a horizontal screw hole (see FIG. 5) formed horizontally so as to communicate at a right angle with the vertical slide slot 111, and into this horizontal screw hole 118 is inserted a fixing screw 119. Accordingly, tightening the fixing screw 119 presses the leg 114 of the slider 113 with the front end of the fixing screw 119 inserted into the slide slot 111 to fix the slider 113 at a predetermined location.

Here, it is preferred that the slide slot 111 and the leg of the slider 113 are formed in an angular shape, preferably a rectangular shape, for the fixing screw 119 to press the slider 113 stably. On the top surface 115 of the slider 113 is formed a through hole 112 for the pin that has passed through the pinhole 15 to be driven through the femur without interference with the slider 113.

Preferably, on the front of the leg 114 of the slider 113 is marked a graduation 117 for measuring the height of ascent and descent of the slider 113. This graduation is for adjusting the height of the slider 113 as much as the offset amount between the horizon perpendicular to the mechanical axis and the protruded portion at the end of the femur. And, in the middle portion of the top surface of the body 110 is formed a support pin 116 having a sharp front end, as shown in FIG. 4, for supporting the guide device by being driven into the bottom end of the femur, or an insert hole 120 for a support bar 116-1 with a sharp front end to be inserted, as shown in FIG. 6. The front end of the support bar 116-1 inserted in the insert hole 120 is driven into the bottom end of the femur.

How to use a femur alignment guide device according to the embodiment configured like above is described below.

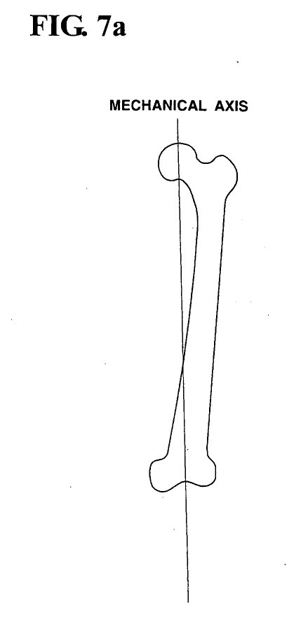

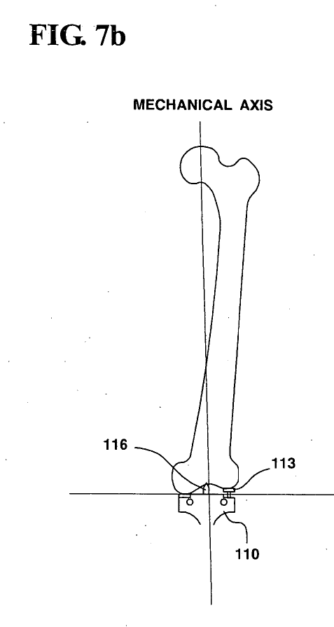

First, as shown in FIG. 7a, obtain a mechanical axis with an image photograph obtained by X-ray photographing the femur. This mechanical axis is obtained by connecting the center of the femur head and the center of femur bottom end each other. Here, since the femur is turned around the mechanical axis, there is an advantage that the mechanical axis is not varied even if the photographing angle is changed.

After obtaining the mechanical axis, obtain a horizon perpendicular to the mechanical axis so as to come in contact with the femur bottom end. At this time, according to the extent of damage to the femur bottom end of the patient, the protruding portion of one side of the femur bottom end is offset at a predetermined interval. Accordingly, measure the offset between the horizon and the protruding portion of the femur bottom end, and adjust the height of the slider 113 of the guide device according to the present invention based on the measured offset amount, and then tighten the fixing screw 119 to fix the slider 113. At this time, the height adjustment of the slider 113 can be seen easily by the graduation 117. And, with the height of the slider 113 adjusted, the guide device is driven into the femur bottom end by using the support pin 116 with a sharp front end or the support bar 116-1, the top surface 115 of the slider 113 comes into close contact with the protruding portions on both sides of the femur bottom end. Accordingly, the centerline of the femur alignment guide device becomes coaxial with the vertical line of the mechanical axis, so that it is possible to align the guide device without deviation along the mechanical axis.

Next, drive the pin P-1 inserted into the pinhole 15 into the femur to fix the femur alignment guide device on the femur. Subsequently, cut the femur front end bone by the femur front end bone cutter 30 and cut the femur bottom end by the femur bottom end bone cutter 40, then the femur bottom end bone can be cut by the guide device coaxially with the mechanical axis.

If a surgical operation is performed like the using the femur alignment guide device according to the present invention, the conventional problems can be solved at a stroke.

Namely, since it is possible to mount the guide device on the femur coaxially with the mechanical axis based only on the mechanical axis, without measuring the angle between the mechanical and anatomical axes as in the conventional method, which could cause an error depending on the photographing angle, it is possible to perform knee arthropolasty without side effects.

Also, since the guide device according to the present invention is mounted on the femur bottom end by adjusting the height of the slider 113 as much as the offset amount, after measuring the offset between the horizon perpendicular to the mechanical axis and the protruding portion of the femur bottom end, it is possible to stably support in close contact the protruding portions on both sides of the femur bottom end of the patient, so deviation of the guide device can be prevented, especially in the process of driving the pin.

Moreover, since it is not necessary to insert a bone cavity bar into the bone cavity as in the conventional method by using the guide device according to the present invention, no problem arises due to input of the bone cavity bar in deviation, and surgical operation is possible even if a hip joint replacement is inserted in the bone cavity. And more than anything else, since there is no problem of damage to bone cavity due to insertion of a bone cavity bar, the patient can be treated quickly without any aftereffects.

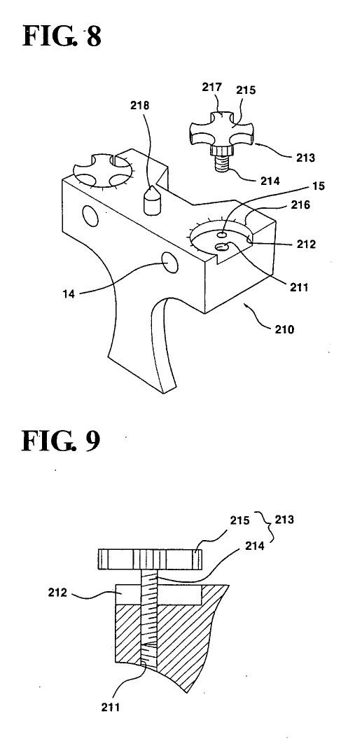

Another embodiment of the top surface height adjusting means of the guide device according to the present invention will be described with reference to FIGS. 8 to 10.

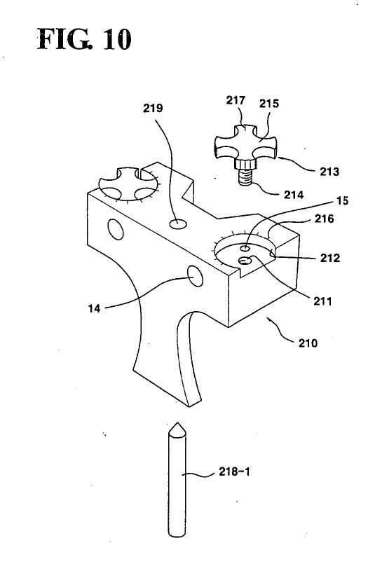

FIG. 8 is a perspective view showing a guide device according to another embodiment of the present invention, FIG. 9 is a sectional view of the top surface height adjusting means shown in FIG. 8, and FIG. 10 is a perspective view showing a modified embodiment of the guide device shown in FIG. 8.

As shown in these drawings, the top surface height adjusting means according to this embodiment includes a body 210, recesses 212 formed on both sides of the body 210 and having vertical screw holes 211; and an adjusting knob 213 screwed to a vertical screw hole 211 of the recess 212 to move up and down.

The recess 212 has a shape with the top surface of the body depressed at a predetermined depth, and in the recess 212 is formed a vertical screw hole 211 for the screw portion 214 of the adjusting knob 213 to be screwed. Accordingly, the screw portion 214 of the adjusting knob 213 is inserted into the vertical screw hole 211 through screwing and the head portion 215 is placed in the recess 212. Here, the top end surface of the adjusting knob 213 becomes a support surface for supporting the bottom end of femur. For example, if the screw portion 214 and the vertical screw hole 211 are of a right-hand screw, the interval to the bottom end of the femur can be adjusted by turning the adjusting knob 213 to the right in the vertical screw hole 211 for descent and to the left for ascent.

Here, the reason for making the shape of the top end of the adjusting knob 213 in a shape of air vane is that the pin P-1 that has passed through the pinhole 15 formed in the recess 212 can be driven into the femur without interference with the top end of the adjusting knob 213. If the pinhole 15 is to be formed in a part other than the recess 212, it is not necessary to form it particularly in a shape of air vane, as the top end of the adjusting knob 213 is not interfered with the pin P-1.

Also, on the top surface of the body 210 is marked a graduation 216 for measuring the height of ascent and descent of the adjusting knob 213, and on the top surface of the adjusting knob 213 is marked an index line 217 indicating the graduation 216. And, in the middle portion of the top surface of the body 110 is formed a support pin 218 having a sharp front end, as shown in FIG. 8, to be driven into the bottom end of femur for supporting the guide device, or an insert hole 219 for a support bar 218-1 with a sharp front end to be inserted, as shown in FIG. 10. The front end of the support bar 218-1 inserted into the insert hole 219 is driven into the bottom end of the femur.

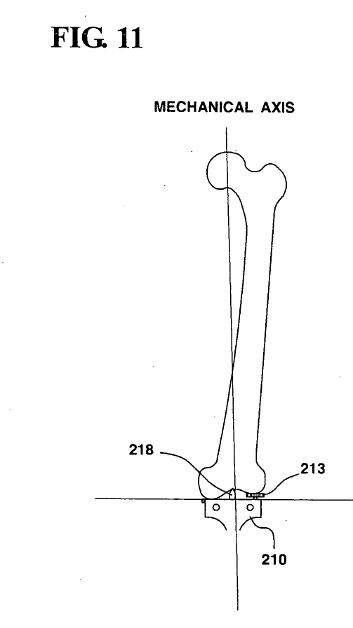

How to use the femur alignment guide device configured like this according to the embodiment will be described with reference to FIG. 11.

First, as shown in FIG. 11, after obtaining a mechanical axis by X-ray photographing, obtain a horizon perpendicular to the mechanical axis so as come in contact with the femur bottom end. Then, measure the offset between the horizon and the protruding portion on one side of the femur bottom end, and adjust the height by turning the adjusting knob 213 of the guide device according to the present invention based on the measured offset amount. At this time, the height of ascent or descent of the adjusting knob 213 can be easily seen by the graduation 216 and the index line 217. And, with the height of the adjusting knob 213 adjusted, the guide device is driven into the bottom end of the femur by using the support pin 218 with a sharp front end or the support bar 218-1 inserted into the insert hole 219, then the top surface 215 of the adjusting knob 213 comes into close contact with both protruding portions of the femur bottom end. Accordingly, the centerline of the femur alignment guide device becomes coaxial with the vertical line of the mechanical axis, so the guide device can be aligned vertically along the mechanical axis.

Next, as described with reference to FIG. 7b, drive the pin P-1 inserted into the pinhole 15 into the femur to fix the femur alignment guide device to the femur. Subsequently, cut the femur front bone by the femur front bone cutter 30 and cut the femur bottom end by the femur bottom end cutter 50, then the femur bottom end can be cut coaxially with the mechanical axis by the guide device.

As described above, the femur alignment guide device according to the present invention can be mounted on the femur always coaxially with the mechanical axis based only on the mechanical axis without measuring the angle between the mechanical and anatomical axes, which is varied according to the photographing angle, so replacement operation without side effects is possible.

Furthermore, since there is no fear of the guide device being in deviation as the both protruding portions on the femur bottom end can be supported wholly in close contact and the guide device can be mounted on the femur vertically along the mechanical axis without inserting a bone cavity bar into the bone cavity, no problems arise due to forming an input hole at the femur bottom end. Another advantage is that even if a hip joint replacement is inserted into bone cavity, surgery can be easily performed and since bone cavity is not damaged, quick recovery is possible and there are no aftereffects.

While the present invention has been described with reference to exemplary embodiments thereof, it will be understood by those skilled in the art that various changes in form and details may be made therein without departing from the scope of the present invention as defined by the following claims.

What is claimed is:

1. A femur alignment guide device comprising:
a body having a pair of mounting holes formed transversely for mounting a femur front bone cutter and a femur bottom end cutter, and a pair of pinholes formed vertically for inserting the pin driven into the femur bottom end; and
a pair of top surface adjusting means mounted on both sides of said body so as to make ascent and descent possible.

2. The femur alignment guide device according to claim 1, wherein said top surface adjusting means comprises a stepped portion formed on both sides of the body of the guide device and having a slide slot with a predetermined depth;
a slider moving up and down by a leg inserted into said slide slot;
a horizontal screw hole formed so as to be penetrated perpendicularly to said slide slot from the back of the body; and
a fixing screw inserted into said horizontal screw hole to press the leg of said slider to fix the slider against the body.

3. The femur alignment guide device according to claim 2, wherein the leg of said slider is marked with a plurality of graduations so as to measure the height of ascent and descent of the slider.

4. The femur alignment guide device according to claim 2, wherein the leg of said slider and slider slot are formed in an angular shape.

5. The femur alignment guide device according to claim 1, wherein said top surface height adjusting means comprises a recess formed on both sides of the body of the guide device and having a vertical screw hole with a predetermined depth; and
an adjusting knob screwed to a vertical screw hole of said recess to slide up and down.

6. The femur alignment guide device according to claim 5, wherein a plurality of graduations are marked around the recess formed on the top surface of said body so as to measure the height of ascent and descent, and an index line indicating said graduations is marked on the top surface of said adjusting knob.

7. The femur alignment guide device according to claim 1 wherein a support pin with a sharp front end driven into the femur bottom end so as to support the guide device is formed in the middle portion of the top surface of said body.

8. The femur alignment guide device according to claim 1 wherein a support insertion hole is formed in the middle portion of the top surface of said body, and a support bar with a sharp end is inserted into this support bar insertion hole.
